(19) United States
(12) Patent Application Publication
(10) Pub. No.: US 2007/0243222 A1
(43) Pub. Date: Oct. 18, 2007

(54) FUNGICIDAL FORMULATION AND METHOD OF USE

(76) Inventors: Carl Lawyer, Springfield, IL (US); Matthew Carl Lawyer, Springfield, IL (US)

Correspondence Address: HAYES SOLOWAY P.C., 3450 E. SUNRISE DRIVE, SUITE 140, TUCSON, AZ, 85718, US

(21) Appl. No.: 11/671,416

(22) Filed: Feb. 5, 2007

Related U.S. Application Data
(60) Provisional application No. 60/765,426, filed on Feb. 3, 2006.

(51) Int. Cl. A61K 33/30; A61K 31/315; A61K 31/355; A61K 9/00; A61P 31/10; A61K 31/44; A61K 33/24

(52) U.S. Cl. 424/404; 424/617; 424/642; 514/188; 514/345; 514/494; 514/645

(57) ABSTRACT

A method for treating onychomycosis of a nail of an animal, which comprises a topical application of a solution to said nail, wherein said solution is comprised of at least one chemical agent capable of chelating iron metal ions

### CROSS REFERENCE TO RELATED APPLICATION

This application claims priority from U.S. Provisional Application Ser. No. 60/765,426, filed Feb. 3, 2006.

### BACKGROUND

1. Technical Field

This invention relates generally to methods and pharmaceutical formulations for the control of fungal disease in animals and humans.

2. Introduction and Discussion of the Prior Art

Fungal infections of the nail, referred to by the terms “nail fungus,” “onychomycosis,” or “tinea unguium,” are common throughout the world. An estimated 2-13% of the population is affected in North America, with at least 15-20% of those aged 40-60 having one or more fingernails or toenails infected. Toenails are much more commonly affected than fingernails. Infections can range from superficial, causing little more than discoloration, to severe, resulting in loss of the nail together with deformities of the surrounding digit. The incidence of onychomycosis has been rising over the past few decades, due to factors such as an increased elderly population, increased participation in vigorous physical activity while wearing moisture-retaining shoes and socks, an increase in the number of HIV infected individuals, an increased incidence of diabetes, and increased use of steroids, antibiotics, and other therapeutics that can suppress immunologic responses to fungi.

While nail fungus is rarely life threatening, it causes significant pain, inconvenience, embarrassment, emotional distress, and limitations to manual performance and ambulation. Individuals with moderate to severe onychomycosis can lose their ability to perform many routine tasks (such as fastening buttons, picking up small objects, walking significant distances) and can lose the ability to perform satisfactorily in their occupations. Due to the unpleasant appearance of their hands or feet, these individuals may become socially self-conscious and embarrassed, and may avoid intimate or other close contact with people. Loss of self-esteem, anxiety, and depression commonly result from moderate to severe cases of fungal nail infection.

At present, topical treatments for nail fungus are rarely effective. Although some oral antifungal therapies have moderate efficacy, they also pose significant risks of toxic reactions, and many patients would prefer local, topical treatments to systemic treatments.

Etiology and Varieties

Onychomycosis is caused most commonly by several species of dermatophytes (parasitic fungi that infect the keratin-rich tissue of the stratum corneum, hair, and nails) and, less commonly, by nondermatophyte molds or by yeasts, primarily of the genus Candida. An estimated 90% of cases are caused by dermatophytes, primarily of the genera Trichophyton, Microsporum, and Epidermophyton, while about 8% are caused by nondermatophyte molds and 2% by yeasts. The causative agent in onychomycosis can rarely be determined by clinical appearance; microscopic examination or culturing is usually required. Furthermore, an infected nail colonized by one species can develop secondary infections by other fungi, yeasts, or bacteria.

Onychomycosis can affect the nail plate, the nail bed (the tissue directly under the nail plate), the nail matrix or nail root (the thickened skin at the base of the nail from which the nail plate develops), the cuticle, the hyponychium (the thickened layer of epidermis beneath the free end of the nail), and the proximal and lateral nail folds (the skin adjacent to the base and sides of the nail).

Factors that contribute to the development of onychomycosis include advanced age, diabetes (which reduces circulation to the extremities), wearing heat- and moisture-retaining footwear, communal bathing, HIV infection, the use of antibiotics or immunosuppressive drugs, trauma to the nail, use of insufficiently cleaned manicure tools, poor overall health, and warm climates.

Onychomycosis can be categorized into several varieties based on clinical appearance. The recognized varieties include:

Distal and lateral subungual onychomycosis (DLSO): This is the most common variety. It usually results from a fungal infection of the skin (usually the plantar skin of the foot) spreading to the nail bed and then to the underside of the nail plate via the hyponychium. The distal and lateral parts of the nail plate become thickened, white, and opaque. In addition, the nail bed becomes hyperkeratotic and onycholysis (separation of the nail plate from the bed, ultimately resulting in loss of the nail) commonly ensues. Paronychia (inflammation of the tissues adjacent to the nail) is also common. Trichophyton rubrum is the most common pathogen.

Endonyx onychomycosis (EO): This is a variety of DLSO in which the fungus spreads directly from the skin to the nail plate rather than to the nail bed. The nail again is thickened, white, and opaque, but there is no evident nail bed hyperkeratosis or onycholysis.

White superficial onychomycosis (WSO): This variety is almost always found on toenails. The surface of an infected nail develops white dots or powdery patches and the nail becomes rough and crumbly. Trichophyton mentagrophytes (T. interdigitale) is the most common cause, though some nondermatophyte molds such as Acremonium, Aspergillus, and Fusarium can also infect the upper surface of the nail plate. The nondermatophyte molds commonly cause black or green nails.

Proximal subungual onychomycosis (PSO): In this least common variety, the fungus first attacks the cuticle and proximal nail fold, and then penetrates the proximal nail plate. The distal part of the nail remains normal.

Candida Onychomycosis (CO): The yeast, nearly always Candida albicans, infects the nail folds (paronychia), the nail plate and surrounding tissues (in chronic mucocutaneous candidiasis), the nail bed, or any combination of these. The entire digit commonly becomes swollen and deformed. Candida may cause onycholysis or it may colonize onycholytic nails (resulting from trauma or another infection). Candida infection associated with paronychia is almost always secondary to trauma to the nail folds.

Total dystrophic onychomycosis (TDO): The entire nail plate is thickened, yellow-brown, and opaque. All the adjacent tissues are affected, and the nail matrix may be permanently damaged, preventing normal nail growth even after the infection resolves. TDO can be the endpoint of any of the other onychomycosis varieties.

A patient with onychomycosis may have one variety or any combination of varieties.

Current Treatments

Onychomycosis is presently treated primarily with oral antifungal agents. Topical agents are rarely effective by themselves, except in mild cases that only affect the distal nail plate. They may, however, be beneficial in combination with oral therapy. In severe cases, the affected nail (and sometimes the nail bed and matrix) is removed surgically or by use of a urea containing formulation; removal of the nail is done in conjunction with oral and sometimes topical therapy. Further details on some of these methods follow.

Oral medications: The preferred therapy for onychomycosis is orally administered treatment with terbinafine (Lamisil™), itraconazole (Sporanox™), or fluconazole (Diflucan™). Terbinafine, an allylamine, is active against dermatophytes, but has considerably less efficacy against nondermatophyte molds and against yeasts. Itraconazole and fluconazole are triazoles that are effective against dermatophytes, nondermatophyte molds, and yeasts. When administered daily, all of these compounds can cause hepatic injury, and monitoring of liver enzymes is required. Pulse therapy (typically, administration one week per month) reduces the risks for hepatic damage, but prolongs the course of therapy from about 6 to 12 weeks to at least several months. Terbinafine has several potentially serious drug interactions, and the triazoles, because they are metabolized using the hepatic cytochrome P450 system, have numerous significant drug and food interactions that prevent their use in many patients. Even though these drugs are the currently preferred treatments for onychomycosis, their cure rates are not high: a recent survey (Arch Dermatol 134(12):1551-1554, 1998) found that standard treatment with terbinafine resulted in disease-free nails in approximately 35-50% of cases, while the rate for itraconazole was approximately 25-40%. Relapse is also common, though precise figures are not available. These oral therapies are nevertheless more effective than topical treatments because they apparently penetrate the nail more quickly and thoroughly, and because they remain in the nail for weeks or months following treatment.

Topical treatments: Topical antifungal treatments are now administered mainly in cases where the fungal infection is restricted to the distal half of the nail plate or in cases in which the patient cannot tolerate oral therapy. Again, their low efficacies appear due mainly to their inability to adequately penetrate the nail. Topical antifungal agents include allylamines (including terbinafine), triazoles (including itraconazole and fluconazole), imidazole derivatives (including ketoconazole, miconazole, clotrimazole, and enconazole), amorolfine, ciclopirox olamine, sodium pyrithione, bifonazole plus urea, and propylene glycol plus urea plus lactic acid.

Existing treatments for onychomycosis are thus of limited efficacy, have high risks for adverse effects and drug interactions, and are time consuming and inconvenient for the patient. Often a treatment that initially appears successful will have a high rate of failure as infection frequently reoccurs. Furthermore, a large majority of onychomycosis patients reportedly would prefer a local, topical treatment to a systemic treatment that carries a significant risk of adverse effects.

The foregoing discussion of the prior art derives from U.S. Pat. No. 6,846,837 which describes a method and formulation for the treatment of onychomycosis that involves a topically applied formulation containing a pharmacologically active base in an amount effective to provide the formulation with a pH in the range of about 7.5 to 13.0, preferably about 8.0 to 11.5, and most preferably about 8.5 to 10.5. The base itself has antifungal activity, but additional antifungal agents reportedly may be incorporated into the formulation as well, in which case the base not only provides antifungal activity but also enhances the permeation of the additional antifungal agent(s) into and through the skin. The formulation may be a lotion, cream, solution, paste, ointment, plaster, paint, bioadhesive, or the like, or may be contained in a tape or in a skin patch comprised of a laminated composite intended for long-term adhesion to the body surface (typically throughout a delivery period in the range of about 8 to about 72 hours) in the affected area.

U.S. Pat. No. 6,664,292 describes topically treating fungal and other microbial infections of the nails with a combination of an optionally substituted lower alcohol and an optionally substituted lower carboxylic acid. The combination functions through maintaining a low viscosity to penetrate the nail and attack the infection.

U.S. Pat. No. 6,296,838 describes topically treating fungal infections of the nails with a herbal extract mixture containing walnut hull extract (taken from Juglans regia), pulverized roots of Nardostachys jatamansi or Vetiveria zizanioides or Catharanthus roseus. The combination of the three extracts produces a synergistic fungitoxic substance.

U.S. Pat. No. 6,264,927 describes a combination of an ethyl ether and an iodine. The ethyl ether component serves to penetrate the nail and the iodine component serves as a fungicide.

U.S. Pat. No. 4,097,590 describes a topical treatment for bacterial and fungal infections of the skin using a combination of sodium fluoride, that acts as the active ingredient to eliminate the bacterial or a fungal infection, and a surface active ingredient, that acts as a wetting agent.

U.S. Pat. No. 6,878,365 describes a topical treatment for toenail fungus using as an active ingredient at least one species selected from 2,2′-(alkyldioxy)bis-(alkyl-1,3,2-dioxaborinane) and 2,2′-oxybis(alkyl-1,3,2-dioxaborinane) and more specifically at least one member selected from the group consisting f 2,2′(1-methyltrimethylenedioxy)bis-(4-methyl-1,3,2-dioxaborinane) and 2,2′-oxybis(4,4,6-trimethyl-1,3,2-dioxaborinane). These compounds are advantageous organo-boron compounds as they can kill Candida albicans without causing significant toxicity to the human it is applied to.

U.S. Pat. No. 6,403,060 describes treating nail fungus by applying a polyurea composition to an affected nail. The therapeutic polyurea serves to provide a means for better penetrating the nail and gaining access to the affected area.

It is known that topical antifungal nail lacquer containing ciclopirox is available for the treatment of mild to moderate onychomycosis that does not involve the lunula. The lacquer is applied once daily to the affected nail, 5 mm of surrounding skin, and to the nail bed, hyponychium, and undersurface of the nail plate if possible. The nail is wiped clean with alcohol once weekly, and the unattached infected part of the nail is removed periodically. Combined results from two randomized, controlled trials suggest that complete resolution occurs in approximately 7 percent of treated patients compared with 0.4 percent using placebo. Hence, only 1 of 15 patients who use the lacquer will have a favorable outcome, and recurrence is common after stopping therapy. Given its low efficacy, except in patients who greatly desire therapy and who are felt to be at high risk for complications of oral therapy, it is generally recommended that patients not use topical therapy for onychomycosis.

Not being bound by theory, we believe that it is the light sensitivity of ciclopirox which explains its low efficacy. The lack of consideration given to protecting ciclopirox from visible light during the manufacturing process, the storage process, and the treatment process accounts for the variability in efficacy. Surprisingly, the implementation of precautions to avoid light exposure, such a storing the ciclopirox in a light-blocking container and/or adding light blocking agents to the lacquer composition, significantly improves upon the efficacy of the conventional treatment with ciclopirox.

### SUMMARY OF THE INVENTION

The present invention is an improvement over the aforesaid and other prior art by providing a novel topically applied treatment for onychomycosis which employs a novel mechanism for treating the offending fungi in humans and animals. More particularly, the present invention provides a novel formulation and methods for treatment of nail fungi and the like, which comprises topically applying to the nail and surrounding areas, an effective amount of a composition comprising a chemical agent capable of enhancing iron starvation, e.g., an iron chelating agent, which may include zinc or a zinc-containing compound alone or in combination with another zinc compound. Some of the iron chelating agents are also able to directly block the iron uptake molecular pumps of the offending fungi by entering and then sticking in and clogging the iron pump channel thus depriving the fungi of essential iron. While not wishing to be bound by theory, the present invention is based on the premise that fungi require essential iron ions for electron transport and oxygen transport. Accordingly, by depriving the fungi of these essential iron ions, and removing the iron from the fungi's micro-environment, the growth and survival of the fungi may be inhibited or significantly reduced. Thus, chelation of iron ions and/or blockade of iron uptake by the fungi deprives the infesting fungi from reproduction and survival.

### DETAILED DESCRIPTION OF PREFERRED EMBODIMENTS

The present invention is based on the topical application of a pharmacologically active iron starvation or iron chelating agent that blocks or deprives fungi of essential iron metal ions. The iron starvation or iron chelating agents (hereinafter “iron chelators”) are applied topically in a pharmaceutically acceptable carrier. As used herein, a “pharmaceutically acceptable carrier” is one that is suitable for use with humans and/or animals without undue adverse side reactions such as toxicity, irritation and allergic response commensurate with the reasonable benefit/risk. Preferred carriers include aqueous-based or alcohol based solvents and nail lacquers. The iron chelating agents also may be applied impregnated on a bandage or the like which is placed in contact with the nail.

The preferred iron chelating agents are those which lack excessive polar groups aside from those required to bind iron, and as a result avoid sequestration by siderophores. Siderophores are a form of iron chelator that are excreted by microbes in order to scavenge the microenvironment for iron. Siderophores can also sequester other iron chelators present in the microenvironment to aid, and they perform this function by interacting with non-iron involved polar groups. It is therefore preferred that the chemical agents used to chelate iron in accordance with the present invention lack these superfluous polar groups and avoid the undesirable effect of aiding microbes to gather iron. Iron chelating agents useful in accordance with the present invention include, but are not limited to:
1) 1,2-diethyl-3-hydroxypyridin-4-one
2) 1,2-dimethyl-3-hydroxypyrid-4-one
3) 1,10-phenanthroline
4) 1-hydroxy-2-pyridone
5) 2-pyrocatechuic acid (DHBA)
6) 3-(N-morpholino)-propanesulfonic acid (MOPS buffer)
7) 3-hydroxy-4-pyridone
8) Adenosine diphosphate-ferric chelate
9) Beta-thujaplicin
10) Caffeic acid (CAF)
11) D-tagatose
12) Deferiprone
13) Deferrichrome
14) Deferoxamine
15) Dipyrithione
16) Ethylenediamine-N,N′-bis(2-hydroxyphenylacetic acid)
17) Exochelins
18) Fe(III)-EDTA
19) Ferrioxamine B
20) Ferrozine
21) Hydroxyethyl starch-deferoxamine conjugate
22) Iminodiacetic resin (Chelex-100)
23) Mycobactin sideropore
24) N-methyl-D-glucamine dithiocarbamate
25) Pentetic Acid
26) Picolinic acid
27) Piroctone
28) Precipitated sulfur
29) Pyochelin
30) Pyoverdin
31) Pyridoxal isonicotinoyl hydrazone
32) Quin2
33) Rhodotoluric acid sideropore
34) Rilopirox
35) Salicylanilide
36) Sodium pyrithione
37) Trisodium HEDTA
38) Zinc pyrithione

An additional iron chelating agent, useful in the present invention, is an iron-chelating agent consisting of ciclopirox in conjunction with a light-blocking agent. Particularly, preferred iron chelating agents useful in the present invention are zinc pyrithione and Chelex-100. Pyrithione is 2-pyridinethiol-1-oxide and is a “bidentate ligand” that has the capacity to complex with metals other than zinc, such as iron. Chelex-100 is a styrene divinylbenzene copolymer containing paired iminodiacetate ions, which act as chelating groups for binding polyvalent metal ions. The particular iron chelating agents acting alone or in combination have been observed to have a potent inhibitory effect on the propagation of Trichophyton rubrum.

In practice, the iron chelating agent is dissolved or suspended in a pharmaceutically acceptable carrier (concentration of 0.0001 to 100 ug/ml) and the resulting composition is applied topically to the area to be treated or the area of infection. Generally, the composition is applied twice daily. The composition may be applied in such formulation as a gel, cream, lotion, paste, ointment or solution. The solutions may be painted onto the skin or applied as a spray or aerosol. Each of these types of formulation may be obtained using conventional procedures known to those skilled in the art and by using known excipients.

A solution may be obtained by diluting a composition of our invention with pharmaceutically acceptable liquids, for example ethanol, n-propanol, isopropanol, propylene glycol, glycerol and polyethers. These diluents clearly may only be incorporated in such a pharmaceutical composition to an extent which causes no precipitation or phase separation, and this can be readily determined by easy experiment.

A gel may be obtained by adding a gelling agent to a composition of the invention as defined hereinabove, and examples of suitable gelling agents are carboxypolymethylene, polyvinylpyrrolidone, polyvinyl acetate, cellulose derivatives such as methyl-, ethyl-, hydroxyethyl-, hydroxypropylmethyl- or sodium carboxymethyl-cellulose, alginates, bentonites and silica.

An ointment may be obtained by dispersing a composition of our invention as defined hereinabove in an essentially immiscible organic phase, for example soft paraffin, optionally in the presence of an emulsifying and/or thickening agent, for example sorbitin monostearate.

A paste may be obtained by thickening a composition of the invention as defined hereinabove with a solid material such as magnesium stearate, zinc oxide, titanium dioxide, a silicate or starch. These and other solid materials capable of forming a plaster with the composition can also be present in “nanoparticle” form, of less than 100 nanometers or one millionth of a millimeter in size, with a resultant change in visibility of the paste on the area of application but still retain any sun-blocking and thickening properties of the coarser material.

A lacquer may be obtained by the addition of a composition of the invention as defined hereinabove to the ingredients, or combination of ingredients, found in common commercially available nail polishes, for example, nitrocellulose, castor oil, amyl stearate, butyl stearate, glycerol, fatty acids, and acetic acids.

Emulsions such as creams or lotions may be obtained by mixing a composition of our invention as defined hereinabove with a suitable emulsifying system. The compositions may also contain other pharmaceutically active ingredients, for example antibacterial agents, other antifungal agents, keratolytic agents or anti-inflammatory antipyretic or vasodilatory agents, as well as conventional excipients such as colors or preservatives as desired.

However, the iron chelating agent also may be bonded to a fabric or dried in place on a fabric, the fabric formed into an antifungal bandage, sock, or lining of footwear.

In other embodiments of the invention, a Chelex 100 resin may be employed as a polymer for forming the lining of footwear or for making fabric of socks or bandages to aid in the treatment or prevention of onychomycosis or diabetic foot ulcers. In another aspect of the invention, the above described antifungal activity of ironstarvation may be further enhanced by the addition of zinc and/or other compounds, such as gadolinium and lanthanum, that displace iron and zinc through transmetallation.

Gadolinium and gadolinium compounds that are useful in the present invention, include but are not limited to gadolinium bromate, gadoteridol, gadobenate dimeglumine, gadoterate, gadopentate, gadodiamide, or motexafin gadolinium. Gadolinium-based contrast agents are widely used to enhance the contrast of images in magnetic resonance imaging procedures. Gadodiamide is marketed under the brand name “Omniscan” and gadobenate dimeglumine is marketed under the brand name “Multihance”. Two categories of gadolinium chelates exist. The first category is comprised of the macrocyclic molecules where the gadolinum cation Gd³⁺ is caged in the pre-organized cavity of the ligand. The second category is comprised of the linear molecules. Gadolinium chelates differ in their thermodynamic stability constants and in their kinetic stability. In general, macrocyclic chelates such as gadoterate (Gd-DOTA) or gadoteridol (Gd-HP-DO3A) are more stable than linear molecules. Even among linear agents, differences can be found. There is evidence that transmetallation can be found in vivo, in the case of certain contrast agents, especially linear chelates, with body cations such as zinc, calcium or iron. Motexafin gadolinium, utilized as a chemotherapeutic agent in cancer treatments, has been shown to disrupt zinc metabolism in human cancer cell lines.

Lanthanum compounds useful in accord with the present invention include, but are not limited to, lanthanum compounds lanthanum bromate, lanthanum nitrate, lanthanum selenate, and lanthanum chloride. The lanthanum cation La³⁺ has been shown to be a positive allosteric modulator (increasing open channel time and decreasing desensitization in a subunit configuration dependent manner), acting through displacement of zinc, at the modulatory sites of native and recombinant GABA receptors.

Thus, the addition of, any of the following agents alone or in combination with each other, gadolinium, gadolinium cations, gadolinium bromate, gadoteridol, gadobenate dimeglumine, gadoterate, gadopentate, gadodiamide, motexafin gadolinium, lanthanum, lanthanum cations, lanthanum carbonate, lanthanum bromate, lanthanum nitrate, lanthanum selenate, lanathum chloride, metallic zinc particles and/or a zinc containing compound selected from zinc acetate, zinc sulfate, zinc gluconate, zinc oxide, zinc chloride, zinc-(hydroxy-)carbonate, or zinc silicate may further enhance the activity of the iron chelating agents, when included in the topical formulation and/or as a component/constituent of an antifungal bandage, socks or shoes.

The preferred embodiment of the agent that may be added to the hereinbefore mentioned composition for enhanced iron starvation is gadodiamide.

The pharmacological activity of the any or all of hereinbefore mentioned pharmaceutical compositions of the invention may be preserved through avoidance of any agents that could react chemically with the active ingredients during the manufacture, preparation, storage, or utilization of the pharmaceutical compositions. For example, activity may be preserved through the addition to the composition of light blocking agents such as, but not limited to, titanium dioxide, octocrylene, para-aminobenzoic acid, salicylates, cinnamates, zinc oxide, oxybenzene, benzophene, benzophene-3, avobenzene, mexoryl, and the like. As another example, useful in accordance with the present invention, storing the pharmaceutical composition in a visible light blocking container may be necessary to maintain its pharmacologic activity, such as a container wrapped in protective aluminum foil or a container composed of visible light blocking materials, similar or the same as those commonly employed for the storage of hydrogen peroxide.

What is claimed is:

1. A method for treating onychomycosis of a nail of a human or animal, which comprises a topical application of a solution to said nail, wherein said solution is comprised of at least one chemical agent capable of chelating iron metal ions, and wherein when the chemical agent comprises ciclopirox, the solution further contains a visible light blocking agent.

2. The method as defined in claim 1, wherein said chemical agent is zinc pyrithione.

3. The method as defined in claim 1, wherein said chemical agent is iminodiacetic acid.

4. The method as defined in claim 1, wherein the solution also contains zinc.

5. The method as defined in claim 4, wherein the zinc comprises metallic zinc particles or a zinc compound selected from the group consisting of zinc acetate, zinc sulfate, zinc gluconate, zinc oxide, zinc chloride, zinc-(hydroxy-)carbonate, or zinc silicate.

6. The method as defined in claim 1, wherein the solution further contains a visible light blocking agent.

7. A composition for treating onychomycosis of the nail of a human or animal which comprises as least one chemical agent capable of chelating iron metal ions, in a pharmaceutically acceptable carrier, and wherein when the chemical agent comprises ciclopirox, the carrier further contains a visible light blocking agent.

8. The composition as defined in claim 7, wherein the chemical agent comprises zinc pyrithione.

9. The composition as defined in claim 7, wherein the chemical agent comprises iminodiacetic acid.

10. The composition as defined in claim 7, further comprising zinc or a zinc containing compound.

11. The composition as defined in claim 10, wherein the zinc or zinc containing compound comprises metallic zinc particles or a zinc compound selected from the group consisting of zinc acetate, zinc sulfate, zinc gluconate, zinc oxide, zinc chloride, zinc-(hydroxy-)carbonate, or zinc silicate.

12. The composition as defined in claim 7, wherein the carrier further contains a visible light blocking agent

13. A method for treating onychomycosis of a nail of a human or animal, which comprises a topical application of a solution to said nail, wherein said solution is comprised of at least one chemical agent capable of enhancing iron starvation, and wherein when the chemical agent comprises ciclopirox, the solution further contains a visible light blocking agent.

14. The method as defined in claim 13, wherein said chemical agent comprises lanthanum.

15. The method as defined in claim 13, wherein the lanthanum comprises cationic lanthanum or a lanthanum compound selected from a group consisting of lanthanum carbonate, lanthanum bromate, lanthanum nitrate, lanthanum selenate, or lanathum chloride.

16. The method as defined in claim 13, wherein said chemical agent comprises gadolinium.

17. The method as defined in claim 16, wherein the gadolinum comprises cationic gadolinium or a gadolinium compound selected from a group consisting of gadolinium bromate, gadoteridol, gadobenate dimeglumine, gadoterate, gadopentate, gadodiamide, or motexafin gadolinium.

18. The method as defined in claim 13, wherein the solution also contains zinc.

19. The method as defined in claim 18, wherein the zinc comprises metallic zinc particles or a zinc compound selected from the group consisting of zinc acetate, zinc sulfate, zinc gluconate, zinc oxide, zinc chloride, zinc-(hydroxy-)carbonate, or zinc silicate.

20. The method as defined in claim 13, wherein the solution further contains a visible light blocking agent.

21. A composition for treating onychomycosis of the nail of a human or animal which comprises at least one chemical agent capable of enhancing iron starvation, in a pharmaceutically acceptable carrier, and wherein when the chemical agent comprises ciclopirox, the carrier further contains a visible light blocking agent.

22. The composition as defined in claim 21, wherein the chemical agent is lanthanum.

23. The composition as defined in claim 21, wherein the lanthanum comprises cationic lanthanum or a lanthanum compound selected from a group consisting of lanthanum carbonate, lanthanum bromate, lanthanum nitrate, lanthanum selenate, or lanathum chloride.

24. The composition as defined in claim 21, wherein the chemical agent comprises gadolinium.

25. The composition defined in claim 24, wherein the gadolinum comprises gadolinium cations or a gadolinium compound selected from a group consisting of gadolinium bromate, gadoteridol, gadobenate dimeglumine, gadoterate, gadopentate, gadodiamide, or motexafin gadolinium.

26. The composition as defined in claim 20, further comprising zinc.

27. The composition as defined in claim 26, wherein the zinc comprises metallic zinc particles or a zinc compound selected from the group consisting of zinc acetate, zinc sulfate, zinc gluconate, zinc oxide, zinc chloride, zinc-(hydroxy-)carbonate, or zinc silicate.

28. The composition as defined in claim 21, wherein the carrier further contains a visible light blocking agent.

29. An article of manufacture for treating and preventing onychomycosis of the nail of an animal which comprises an iron chelating agent and/or a chemical agent capable of enhancing iron starvation on a solid carrier, and wherein when the chemical agent comprises ciclopirox the solid carrier further contains a visible light blocking agent.

30. An article of manufacture as defined in claim 29, wherein the solid carrier comprises a bandage.

31. An article of manufacture as defined in claim 29, wherein the solid carrier comprises a fabric.

32. An article of manufacture as defined in claim 29, wherein the solid carrier comprises a lining in footwear.

33. An article of manufacture as defined in claim 29, wherein the solid carrier comprises a sock or stocking.

34. An article of manufacture as defined in claim 29, wherein the solid carrier further comprises zinc.

35. The article of manufacture as defined in claim 34, wherein the zinc comprises metallic zinc particles or a zinc compound selected from the group consisting of zinc acetate, zinc sulfate, zinc gluconate, zinc oxide, zinc chloride, zinc-(hydroxy-)carbonate, or zinc silicate.

36. The article of manufacture as defined in claim 29, wherein the solid carrier further contains a visible light blocking agent.
